(19) United States
(12) Patent Application Publication
(10) Pub. No.: US 2010/0247674 A1
(43) Pub. Date: Sep. 30, 2010

(54) MAGNOTHERAPY PRODUCTS

(76) Inventors: Derek PRICE, Wiltshire (GB); Nyjon Eccles, London (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/712,978

(22) Filed: Feb. 25, 2010

Related U.S. Application Data
(60) Provisional application No. 61/155,333, filed on Feb. 25, 2009.

(51) Int. Cl. A61K 33/24; A61K 33/00; A61P 35/00

(52) U.S. Cl. 424/617; 424/600

(57) ABSTRACT

The present invention relates to the use of magnets in the treatment of cancer wherein the magnetic product comprises a magnet having positive and negative poles, the magnet having an additional metallic directional which distorts the magnetic field distribution around the magnet so as to attenuate the magnetic field in the vicinity of the positive pole of the magnet. The invention also relates to methods of treatment using those magnets.

### FIELD OF INVENTION

The present invention relates to the use of magnetic products, i.e. products including one or more magnets, in the treatment of cancer and/or menopause.

### BACKGROUND

Magnotherapy describes the practice of placing magnets adjacent an animal or human body in order to alleviate symptoms or to enhance performance of the animal or human. The mechanism by which magnotherapy works has to date not been understood or confirmed by the scientific community. A discussion of magnotherapy can be found in the book “Magnetic Health, Modern Day Healing with Magnets” by D R Price (ISBN 0953679705).

Menopause indicates the end of reproductive capacity of women and arises from the cessation of ovarian function. Menopause is a gradual process that, for most women, occurs between the ages of 47 and 55 years. It is confirmed by the absence of menstrual periods for 12 consecutive months and excludes other obvious pathologic or physiologic causes. The peri-menopause, a time of changing ovarian function, precedes the final menses by 2 to 8 years. The clinical manifestations of this transition to menopause are not well understood; however, some symptoms such as hot flashes/flushes, begin in the peri-menopause and increase in occurrence as women progress through the menopause. The specific symptoms associated with menopause vary among cultures, race/ethnicity, social groups, and persons. However, symptoms include: hot flashes, irregular periods, emotional responses, changes in sexual relationship, anxiety, feelings of doom, sudden weight gain, increased muscle tension, mood swings, marked fatigue, vaginal dryness, trouble sleeping, urinary incontinence, breast tenderness/soreness, stomach problems (e.g. bloating and/or gas), irritability, loss of libido/sex drive, inability to concentrate, painful and sore muscles and/or disturbing lapses of memory.

Hormone Replacement Therapy (HRT) is currently used to alleviate the symptoms experienced by menopausal women. Hormones used in HRT usually include one or more estrogens. Sometimes, HRT further includes one or more of a progesterone, a progestin and testosterone. However, the use of HRT is associated with risks such as venous thromboembolism and endometrial cancer (Grady et al, 1995), and it is also thought to be associated with increased risks of breast cancer (Collaborative Group on Hormonal Factors in Breast Cancer. Breast cancer and hormone replacement therapy, 1995) and ovarian cancer (Rodriguez et al, 2001).

It is understandable, given the above reported side effects, that there has been more reluctance recently on the part of women to take HRT and more caution on the part of doctors to issue it so freely.

Menopause is sometimes associated with an increased risk of cancer. IGFBP-3 is a blood marker that is currently thought to have a role in cancer prevention and induction of apoptosis (programmed cell death) in cancer cells. IGFBP-3 is thought to be a tumour suppressor. IGFBP-3 is a mediator of growth suppression signals and a putative tumour suppressor gene. The growth suppression mechanisms of IGFBP-3 have not been well clarified. IGFBP-3 mRNA is more abundantly expressed in hypoxia-related inflammatory angiogenesis and recent in viva data suggest that IGFBP-3 has direct, IGF-independent inhibitory effects on angiogenesis. IGFBP-3, the principal carrier of IGFs in the circulation, contributes to both endocrine and autocrine/paracrine growth control; it can be induced by Growth Hormone (GH), cytokines, retinoic acid, and tumour suppressors. Induction of IGFBP-3 by, the well known tumour suppressor, p53 has been shown in various models that directly manipulate p53 activity.

The consensus of scientific research suggests that a reduced level of IGFBP-3 results in increased vulnerability to certain cancers including hepatocellular carcinoma, breast cancer, prostate cancer, lung cancer, melanoma, head and neck cancers. Indeed, IGFBP-3 has been reported to be down-regulated in cancer tissue.

Normal breast epithelial cells are known to exert an apoptotic effect on breast cancer cells, resulting in a potential paracrine inhibition of breast tumour development. IGFBP-3 and maspin are pro-apoptotic factors produced by normal breast epithelial cells. Immunodepletion of IGFBP-3 and maspin completely abolished the normal cell-induced apoptosis of cancer cells. Together these results indicated that normal breast epithelial cells can induce apoptosis of breast cancer cells through IGFBP-3 and maspin. These findings provide a molecular hypothesis for the long observed inhibitory effect of normal surrounding cells on breast cancer development.

In general the anti-cancer activities of IGFBP-3 are likely to be due to its tumour suppressor and apoptotic activities as mentioned above. IGFBP-3 seems to act as a tumour suppressor gene in several human cancers examined. Subramanian et al (Anticancer Res. 2007 September-October; 27(5B): 3513-8) provides evidence for a tumour suppressive function of IGFBP-3 in human breast cancer. Further evidence is provided by Li et al (Prostate. 2007 Sep. 1; 67(12): 1354-61. Hypoxia-inducible factor-1alpha (HIF-1alpha) gene polymorphisms, circulating insulin-like growth factor binding protein (IGFBP)-3 levels and prostate cancer) in relation to prostate cancer.

Although the above mentioned, and many other, studies provide evidence that IGFBP-3 is associated with cancers, to date there has been no indication of a suitable manner in which to deliver IGFBP-3 to a patient and/or to induce increased IGFBP-3 expression by a patient.

What is required is a method of delivering IGFBP-3 to a patient and/or inducing increased expression of IGFBP-3 in a patient. Further required is an alternative treatment of menopause.

### SUMMARY OF THE INVENTION

Surprisingly, the inventors have found that application of a magnetic product such as a south-seeking magnet to a living body leads to changes in the level of one or more blood hormones or other factors that are consistent with a potential anti-cancer action. The application of such a magnetic product, surprisingly, increases the level of IGFBP-3 in a subject to which the product is applied.

Furthermore, application of such a magnetic product to a living body results in a reduction in the symptoms experienced by menopausal women.

Therefore, a first aspect of the invention relates to the use of a magnetic product to prevent and/or to treat cancer wherein the magnetic product comprises a magnet having positive (north seeking) and negative (south seeking) poles, the magnet having an additional metallic element, such as a directional plate, which distorts the magnetic field distribution around the magnet so as to accentuate the magnetic field in the vicinity of the negative pole of the magnet.

The element or directional plate functions to increase the magnetic field in the vicinity of the negative pole. It may also act to attenuate the magnetic field in the vicinity of the positive pole.

The first aspect of the invention also provides a method of preventing and/or treating cancer comprising applying a magnetic product to a human body wherein the magnetic product comprises a magnet having positive and negative poles, the magnet having an additional metallic element. such as a directional plate, which distorts the magnetic field distribution around the magnet so as to attenuate the magnetic field in the vicinity of the positive pole of the magnet.

Notably the changes in the level of one or more blood hormones include a significant increase in blood estriol and in IGFBP-3. Therefore, the use and/or method may cause levels of estriol in the blood to increase and/or levels of IGFBP-3 to increase.

A second aspect of the invention relates to the use of a magnetic product to increase the level of IGFBP-3 in a subject, wherein the magnetic product comprises a magnet having positive and negative poles, the magnet having an additional metallic element, such as a directional plate, which distorts the magnetic field distribution around the magnet so as to accentuate the magnetic field in the vicinity of the negative pole of the magnet.

The second aspect of the invention also provides a method of increasing the level of IGFBP-3 in subject comprising applying a magnetic product to the subject wherein the magnetic product comprises a magnet having positive and negative poles, the magnet having an additional metallic element, such as a directional plate, which distorts the magnetic field distribution around the magnet so as to accentuate the magnetic field in the vicinity of the negative pole of the magnet.

The subject may be a female, such as a menopausal female.

The cancer may be a female specific cancer such as a breast cancer. Alternatively, or in addition, the cancer may be a hepatocellular carcinoma, prostate cancer, lung cancer, melanoma, head cancer or neck cancer.

Since the subject having cancer may be menopausal, application of the magnetic product may treat and/or prevent menopause and cancer at the same time.

The ranges of magnetic strength are optimised for clinical effect while balancing the requirements of magnet size, weight and attraction of ferrous material.

Use of the magnetic product to reduce symptoms of menopause and/or to prevent or treat cancer reduces and/or eliminates the requirement for drugs, such as HRT and/or anti-cancer drugs, may therefore reduce the overall cost of treatment of a subject. Furthermore, there is no need to determine the level of drugs required for a given subject. The use also reduces and/or eliminates the risk of side effects associated with such drugs.

The magnetic product is applied to a human, for example to the pelvic region of a human.

In some embodiments, the magnetic product is applied to the body for at least 2, 4, 6, 8, 10, 12, 14, 16, 18, 20, 22 or 24 hours per day. Furthermore, the magnetic product may be applied to the body for from 7 to 28 days per month, for example 7 to 14 days, 7 to 21 days, 14 to 21 days, 14 to 28 days or 21 to 28 days per month. Preferably the magnetic product is applied for at least ten days, being worn daily throughout the menopause.

A magnetic product may comprise a single magnet. However, the magnetic product may comprise more than one magnet, for example 2, 3, 4, 5, or 6 magnets.

The magnetic element may comprise a directional plate.

The strength of magnet may be varied in accordance to the condition being treated. For example, the strength may be above 750 G, for example from 750 G to 5,000 G, such as from 800 G to 3,600 G as measured at the surface of the magnetic product.

In some embodiments, the magnet has a strength of about 2,000 G, for example from about 1,800 G to about 2,800 G. The magnet may comprise a directional plate to enhance and focus the magnetic field towards the user. The ranges disclosed are optimised for clinical effect while balancing the requirements of magnet size, weight and attraction of ferrous material.

In some embodiments, one or more of the magnets comprises neodymium.

### BRIEF DESCRIPTION OF THE DRAWINGS

Experimental data relating to the invention will now be described, by way of example only, with reference to the accompanying figures, FIGS. 1 to 40.

FIGS. 1 to 23 are box and whisker plots relating to Appendix 1. Figures Ito 23 show symptoms of menopause measured at time points. The symptoms analysed are summarised below.

FIGS. 24 to 39 are box and whisker plots relating to Appendix 2. FIGS. 24 to 39 show hormone and other markers measured at time points. The hormones and others markers analysed are detailed below.

FIG. 40 shows the LadyCare magnetic device.

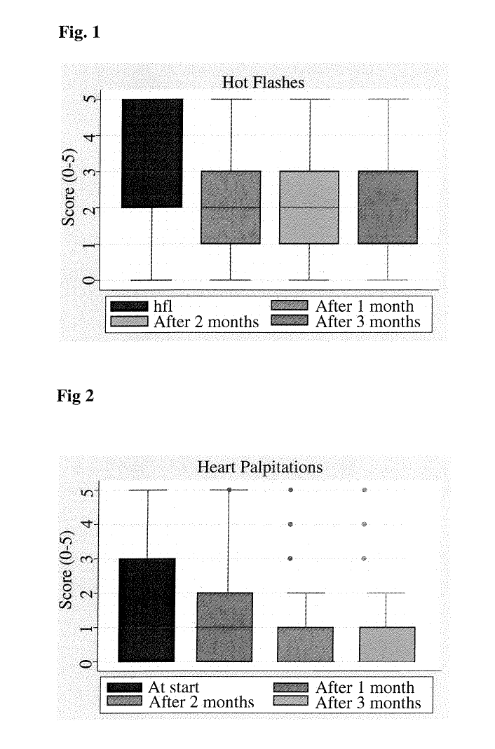

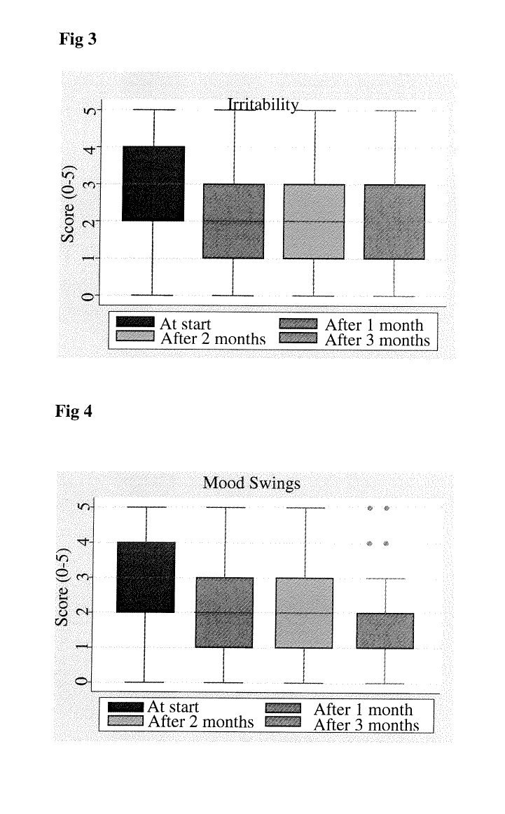

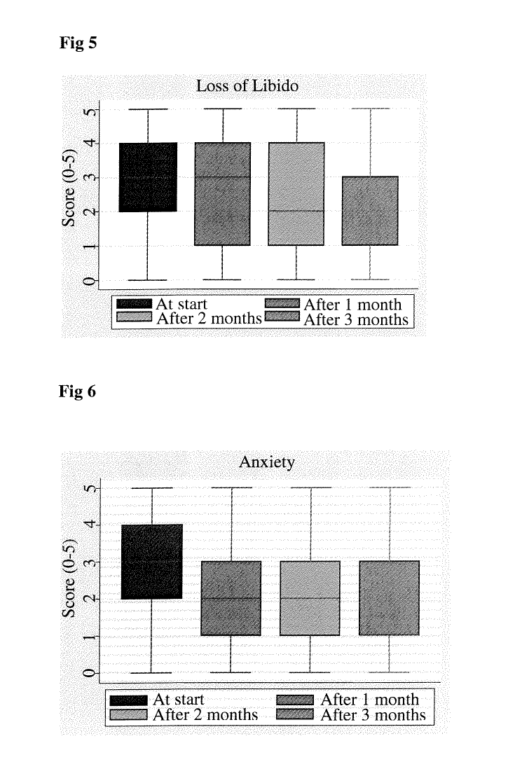

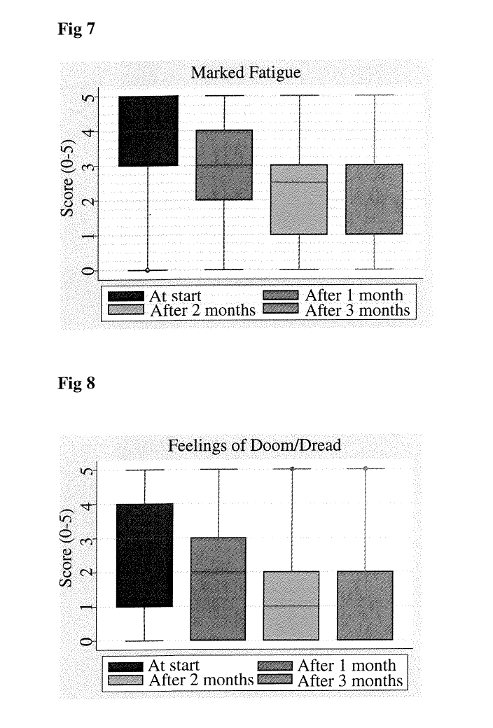

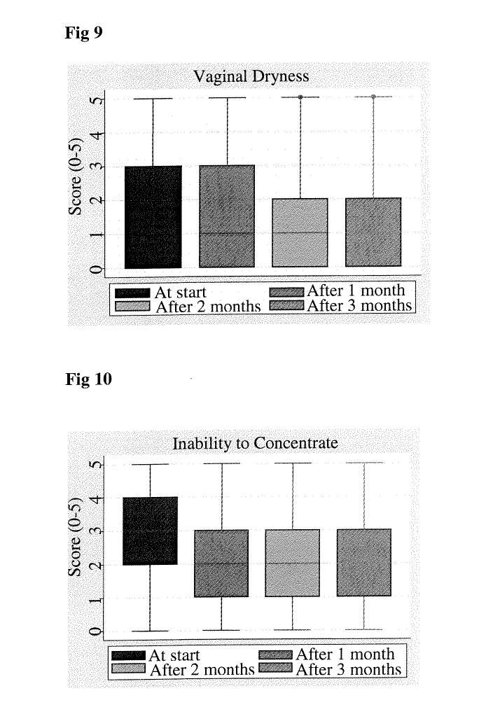

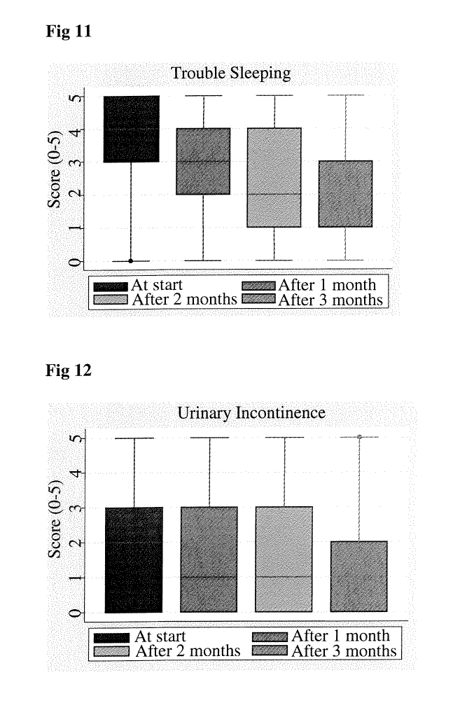

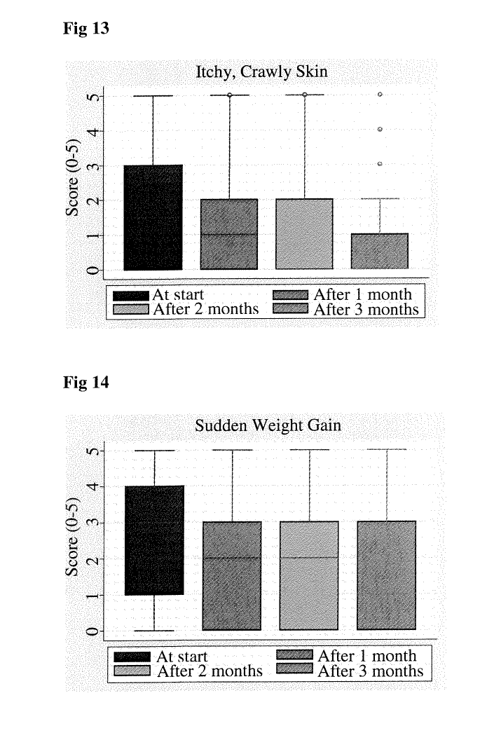

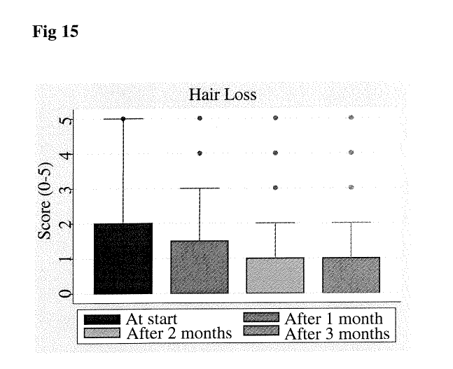

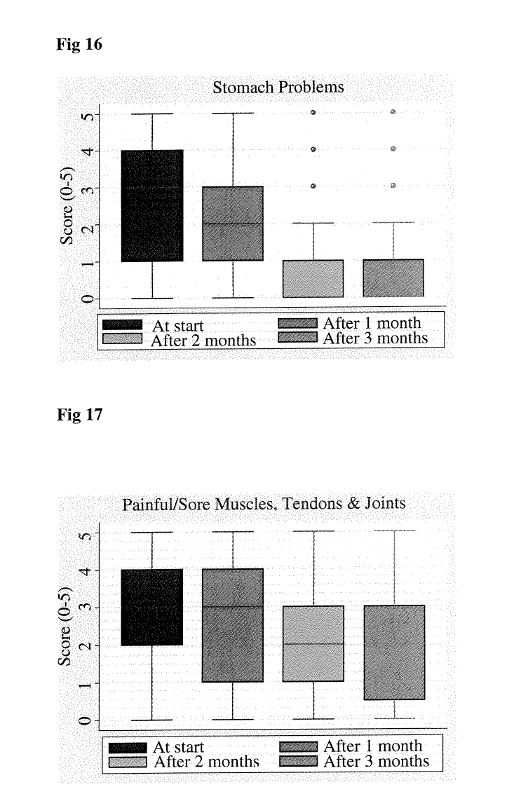

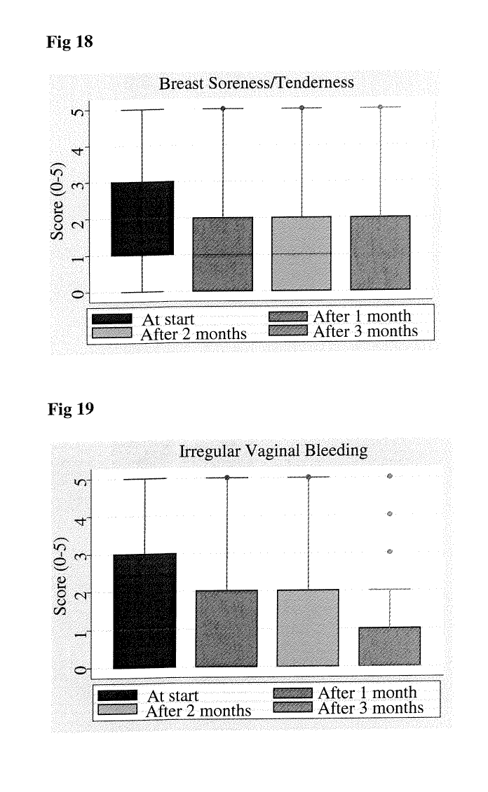

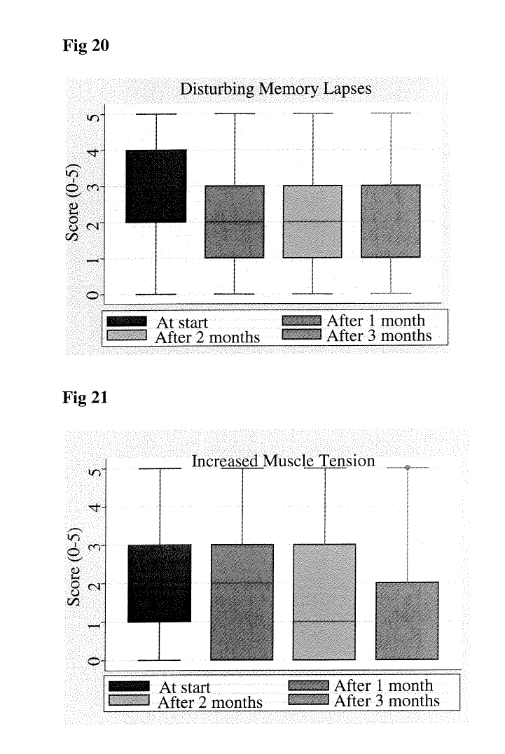

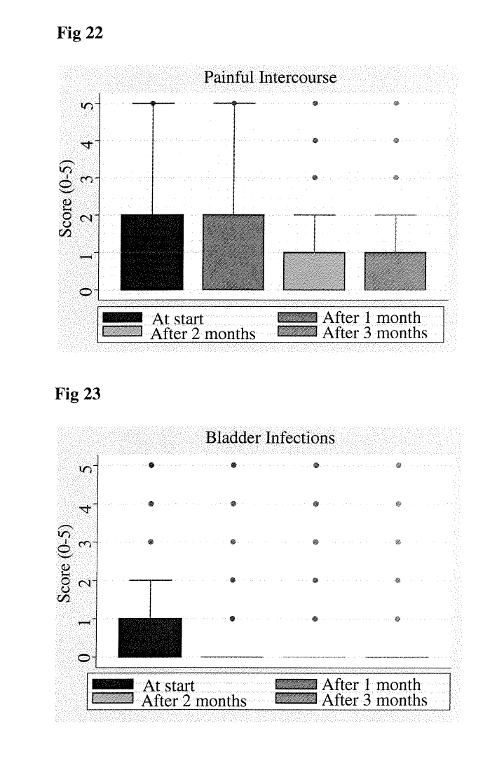

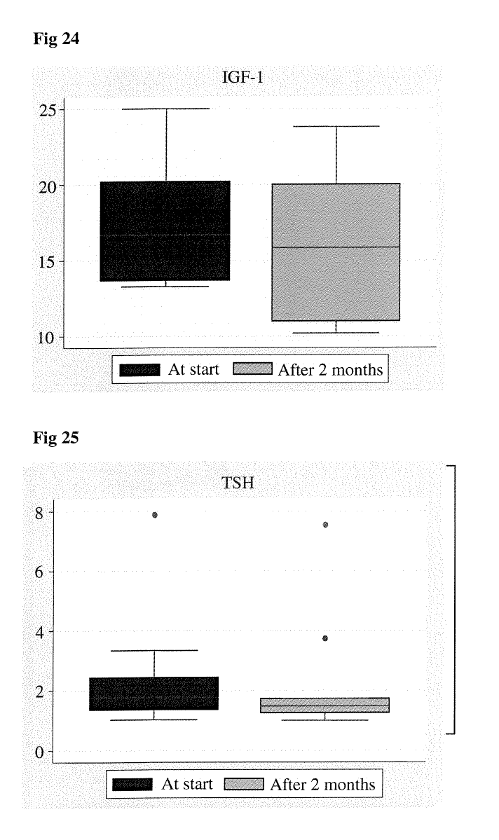

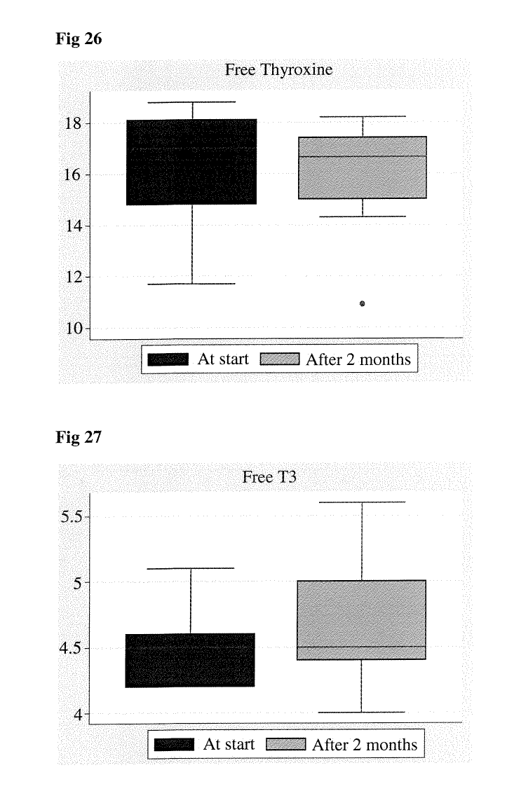

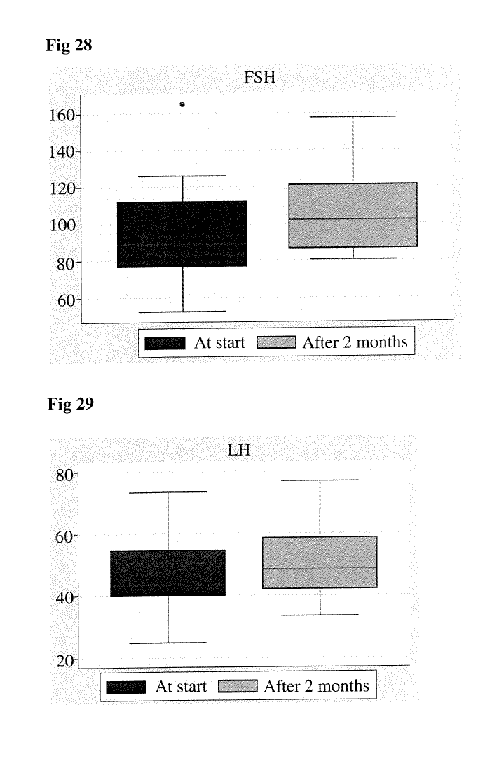

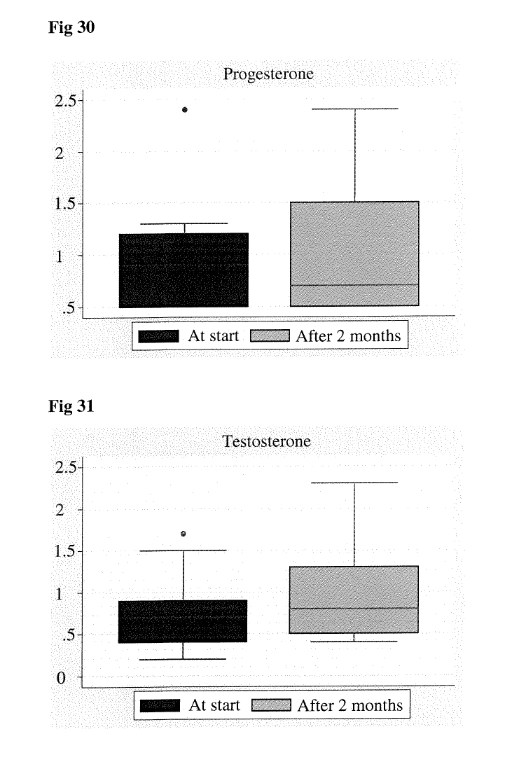

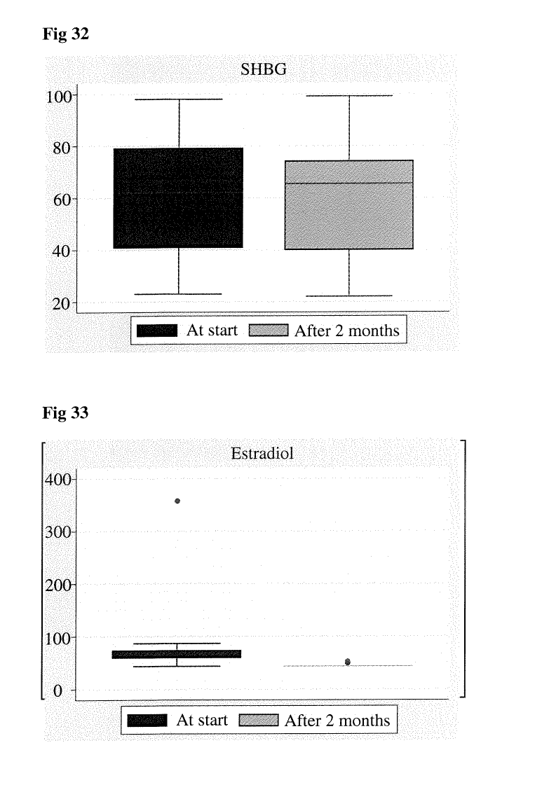

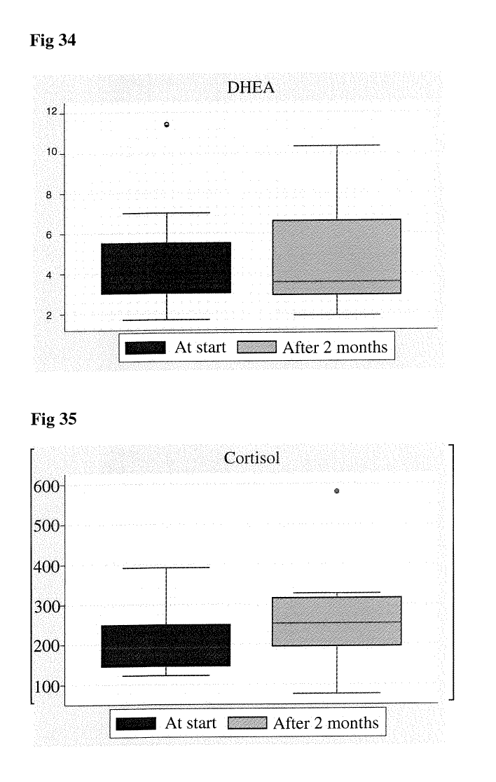

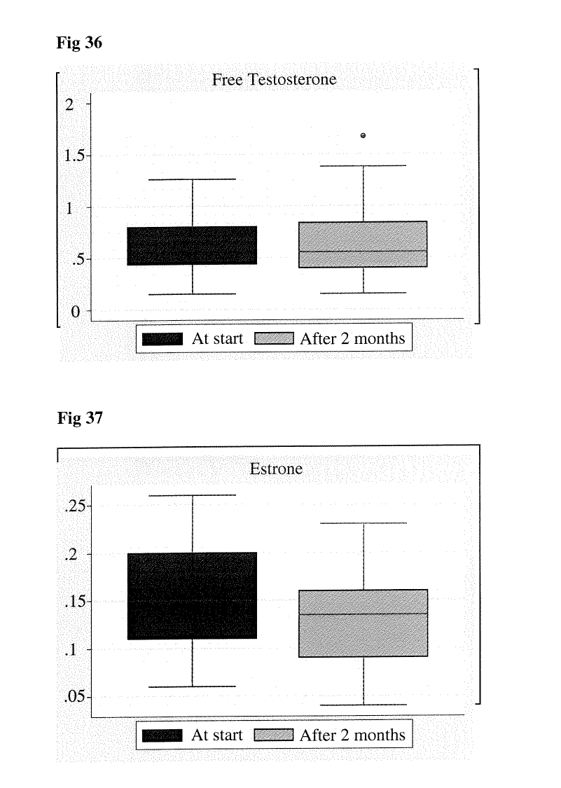

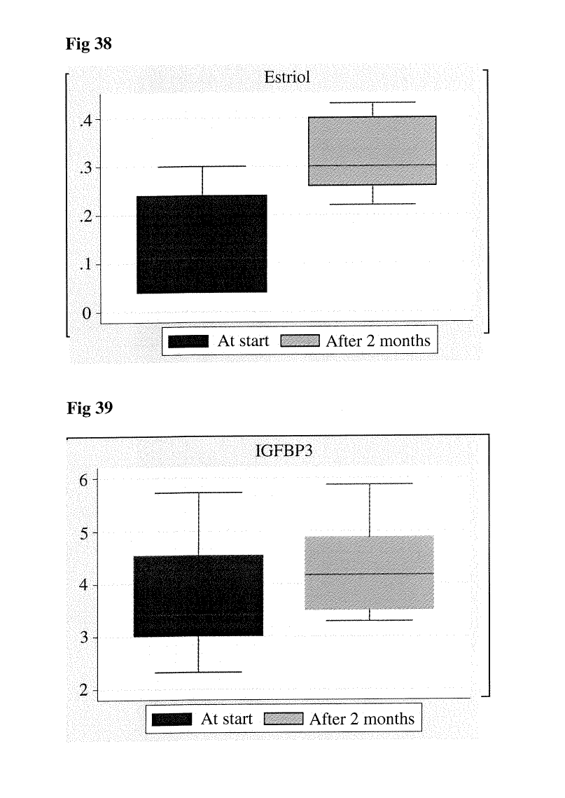

In the figures, ‘S’ refers to a measurement taken at the start of the treatment, ‘1’ refers to a measurement taken one month after the start of the treatment and ‘2’ refers to a measurement taken two months after the start of the treatment.

| | | | |
|---|---|---|---|
| FIG. 1: | Hot flushes | FIG. 2: | Heart palpitations |
| FIG. 3: | Irritability | FIG. 4: | Mood swings |
| FIG. 5: | Loss of libido | FIG. 6: | Anxiety |
| FIG. 7: | Marked fatigue | FIG. 8: | Feelings of doom/dread |
| FIG. 9: | Vaginal dryness | FIG. 10: | Inability to concentrate |
| FIG. 11: | Trouble sleeping | FIG. 12: | Urinary incontinence |
| FIG. 13: | Itchy, crawly skin | FIG. 14: | Sudden weight gain |
| FIG. 15: | Hair loss | FIG. 16: | Stomach problems |
| FIG. 17: | Sore muscles | FIG. 18: | Breast soreness/tenderness |
| FIG. 19: | Irregular vaginal | FIG. 20: | Lapses of memory |
| | bleeding | | |
| FIG. 21: | Muscle tension | FIG. 22: | Painful intercourse |
| FIG. 23: | Bladder infections | FIG. 24: | IGF-1 |
| FIG. 25: | TSH | FIG. 26: | Free thyroxine |
| FIG. 27: | Free T3 | FIG. 28: | FSH |
| FIG. 29: | LH | FIG. 30: | Progesterone |
| FIG. 31: | Testosterone | FIG. 32: | SHBG |
| FIG. 33: | Estradiol | FIG. 34: | DHEA |
| FIG. 35: | Cortisol | FIG. 36: | Free testosterone |
| FIG. 37: | Esterone | FIG. 38: | Estriol |
| FIG. 39: | IGFBP-3 | | |

### Experimental Data

### (a) Menopausal Symptoms (‘Descriptive Data’)

In order to analyse the effect of the magnetic device on the symptoms experienced by menopausal women, 508 menopausal women users of the magnetic device described above were surveyed. Statistically significant reductions in menopausal symptoms were reported after 1, 2 and 3 months by the women.

508 subjects were studied. The subjects rated their menopause symptoms on a 5 point scale (where 1 is low and 5 is high) at 1, 2 and 3 months after commencing use of the magnetic device.

23 of the most common menopause symptoms were rated. The average age of the subjects 49.7 years (±0.199). The median duration of menopause was 2.1 years (1.3-2.5 quartile ranges). It was found that use of the magnetic device caused 21 of the 23 symptoms to reduce significantly (p<10−4). The other 2 symptoms were rated as zero across the group prior to commencing us of the magnetic device, therefore no change could be determined in this study.

The following symptoms were reduced by 50 to 67%: anxiety, feelings of doom, sudden weight gain, increased muscle tension, mood swings, marked fatigue, vaginal dryness, trouble sleeping, urinary incontinence, breast tenderness/soreness, stomach problems (bloating and gas) were reduced by 100%.

The following symptoms were reduced by 33%: hot flashes, irritability, loss of libido/sex drive, inability to concentrate, painful sore muscles and disturbing lapses of memory.

Table A summarises the reported level of reduction in all the 23 symptoms in the group.

8.1% of the subjects had had a hysterectomy/oopherectomy. This did not seem to affect the response to the use of the magnetic device. There was no statistical difference in reduction of symptoms between those who still had a uterus and those that did not.

7.1% of the subjects were taking HRT. This did not seem to affect the response to the use of the magnetic device. There was no statistical difference in reduction of symptoms between those who were taking HRT and those that were not.

19.1% of the subjects lost weight through use of the use of the magnetic device. Median weight loss was 14 pounds (8-17 pounds) over the 3 months (i.e. about 6.5 kg (about 3 to 8 kg)).

29% of the subjects reported a return of their periods after using the magnetic device. The subjects experienced one period and it was generally very light. This was experienced by subjects who were in the early stages of the menopause.

### (b) Hormones and Other Markers (‘Hormone Profile Data’)

A further study was carried out in order to analyse whether the magnetic device affected the hormone levels of the wearers. The study group consisted of 10 menopausal women (not taking HRT). The key findings are summarised in Table B. Results are shown in FIGS. 24 to 39 and the raw data is shown in like-numbered Tables 24 to 39 (Appendix 2). Note that the nomenclature d XX in these tables refers to the difference in measurement of hormones at 2 months compared to baseline (start of the trial).

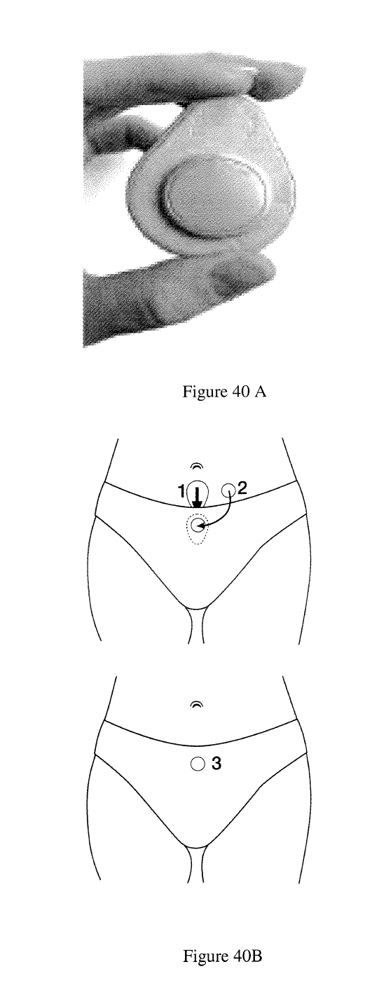

FIG. 40 shows the LadyCare magnetic device is designed for attachment to the underwear by magnetic force. LC is plastic coated and is comprised of two parts. The pear-shaped piece is worn inside of the ladies' underwear, directly against the pelvis. The LC contains a 20-mm×5-mm neodymium iron-boron magnet within the pear-shaped piece. The second part is a circular plastic case that contains a 20-mm×1-mm neodymium iron-boron magnet with a 20-mm×2-mm 400-grade stainless steel directional plate adherent to its outside. This second part positioned on the outside of the underwear (as shown in B) attaching securely with the force of the magnetic field. This directional plate increases the magnetic power of the north pole (standard scientific notation) of the magnetic field into the body to 2700 gauss (surface measurement made by manufacturer at the body surface of the magnet (both parts) using a gauss meter.

Blood hormone analysis was carried out for 10 women with menopausal symptoms, but not taking HRT. The analysis was of a full blood female hormone panel (TDL labs, London) measured firstly at baseline and then repeated 2 months after daily wear of the magnetic device (applied to the pelvic region). Symptoms were also measured on subjective scales exactly as they had been on the previous larger survey of 508 women. Consents for blood draw and participation in the research were obtained from all women.

Statistically significant differences and trends to statistically significant differences in symptoms were noted in this sample of 10 women. The differences and trends identified through hormone analysis confirm the trend that had already been observed in symptom relief in the aforementioned larger survey of 508 women with menopause symptoms.

In these 10 subjects, the symptom analysis indicated trends towards significant reductions in: heart palpitations, irritability, anxiety. marked fatigue, vaginal dryness, bloating and stomach problems. Furthermore, the hormone analysis showed significant reductions in: loss of libido, trouble sleeping and night sweats.

TABLE B
Effect of wearing the magnetic device
on hormone levels of menopausal women.

| | | Affect |
|---|---|---|
| | | of magnetic product on |
| Abbreviation | Hormone | hormone level |
| IGF1 | Insulin-like Growth Factor 1 | No change |
| TSH | Thyroid Stimulating Hormone | No change |
| — | Free Thyroxine | No change |
| Free T3 | Free Tri-iodothyronine | No change |
| FSH | Follicle Stimulating Hormone | No change |
| LH | Luteinising Hormone | Significant increase |
| | | (median 5 point |
| | | [12%] increase) |
| — | Progesterone | No change |
| — | Testosterone | No change |
| SHBG | Sex hormone binding globulin | No change |
| — | Estradiol | Significant reduction |
| | | (median 21 points |
| | | [32%] decrease) |
| DHEA | Dehydroepiandrosterone | No change |
| — | Cortisol | No change |
| — | Free testosterone | No change |
| — | Estrone | No change |
| — | Estriol | Significant increase |
| | | (median 172% increase) |
| IGFBP-3 | Insulin-like growth factor | Significant increase |
| | binding protein 3 | (median 22% increase) |

As shown in Table B, a significant reduction in the level of estradiol and significant increases in the levels of estriol and IGFBP-3 were observed.

A small (12%), but significant, increase in luteinising hormone (LH), one of the main pituitary hormones responsible for ovarian stimulation was observed. The LH level increases after menopause due to lack of negative feedback on the pituitary from reducing levels of estrogen. It is uncertain whether the small increase observed in this study may be as a result of direct stimulatory effect on the pituitary or whether it is due to the reduction in the level of estradiol (see Table B). Estradiol is one form of estrogen. The reduction in estradiol may result in a reduced level of negative feedback on the pituitary and hence an increase in the level of LH. The reduction in estrogen levels per se cannot account for the reduction in menopause symptoms.

There are three main estrogens: estrone, estradiol and estriol. It is generally thought that estriol is a less active estrogen than estradiol and that estriol seems to have protective effects in particular against hormone-related cancers, for example breast cancer. Estradiol can be converted to estriol in the body so it is possible that this conversion pathway is somehow activated by static magnetic fields, causing a rise in estriol but a fall in estradiol. Small doses of estriol have been demonstrated to remit or arrest breast cancer in menopausal women (Lemon HM et al (1975) Cancer Res 35: 1341-1352).

### (c) Comparison of Descriptive Data and Hormone Profile Data

The descriptive data (see Table A) and hormone profile data (see Table B) were analysed. As shown in Table C, there was a good correlation between the descriptive data and hormone profile data. This strongly suggests that the magnetic product is responsible for the improvement in symptoms experienced by the subjects of these studies.

TABLE C
Correlation between changes in symptoms reported by menopausal
women and changes in hormone levels measured in menopausal women.

| | | | Statistically |
|---|---|---|---|
| | | | significant |
| Hormone | Symptom | Correlation | result |
| IGF | Hot flushes | 0.6 | |
| | Urinary incontinence | 0.8 | * |
| TSH | Anxiety | −0.6 | |
| | Trouble sleeping | −0.8 | * |
| Thyroxine | Feelings of doom | 0.6 | |
| | Night sweats | −0.7 | |
| | Painful intercourse | −0.7 | |
| T3 | Anxiety | −0.7 | |
| | Itchy skin | −0.7 | |
| | Painful intercourse | −0.7 | |
| FSH | Anxiety | −0.8 | * |
| | Itchy skin | −0.6 | |
| LH | Hot flushes | 0.6 | |
| | Anxiety | −0.7 | |
| | Memory lapses | −0.6 | |
| Progesterone | Marked fatigue | −0.8 | |
| | Vaginal dryness | −0.7 | |
| | Trouble sleeping | −0.6 | |
| | Bloating | −0.7 | |
| | Weight gain | −0.8 | * |
| | Stomach problems | −0.7 | * |
| | Sore muscles | −0.7 | |
| | Breast soreness | 0.7 | |
| Testosterone | Mood swings | −0.6 | |
| | Anxiety | −0.6 | |
| | Sore muscles | −0.7 | |
| SHBG | Itchy skin | −0.6 | |
| | Stomach problems | −0.6 | |
| Estrogen | Anxiety | 0.7 | |
| | Itchy skin | 0.7 | |
| DHEA | Marked fatigue | −0.7 | |
| | Bloating | −0.8 | |
| | Stomach problems | −0.6 | |
| Cortisol | Vaginal dryness | −0.8 | |
| | Free testosterone | −0.6 | |
| | Vaginal dryness | −0.6 | |
| Estrol | Anxiety | 0.7 | |
| Estriol | Anxiety | 0.7 | |
| | Itchy skin | 0.7 | |
| IGFBP-3 | Sore muscles | −0.7 | |

As shown in Table B, use of the magnetic product induced a significant increase in the level of IGFBP-3 in the subjects. Thus the surprising finding was made that magnetic product may be useful in the prevention and/or treatment of cancers.

TABLE A
The data are also shown in the figures.
Appendix 1. SYMPTOMS - ‘DESCRIPTIVE STATISTICS’

| | | | | | |
|---|---|---|---|---|---|
| | | | 25^{th} | | 75^{th} |
| N | mean | se(mean) | percentile | median | percentile |

TABLE A.1: HOT FLUSHES: (SEE ALSO FIG. 1)

| | | | | | | |
|---|---|---|---|---|---|---|
| Hot | | | | | | |
| flashes | | | | | | |
| At | 508 | 3.1 | 0.072 | 2 | 3 | 5 |
| start | | | | | | |
| After 1 | 508 | 2.3 | 0.069 | 1 | 2 | 3 |
| month | | | | | | |
| After 2 | 508 | 2.0 | 0.067 | 1 | 2 | 3 |
| months | | | | | | |
| After 3 | 508 | 1.8 | 0.065 | 1 | 2 | 3 |
| months | | | | | | |
| At | 508 | 0.9 | 0.052 | 0 | 1 | 1 |
| start - | | | | | | |
| After 1 | | | | | | |
| month* | | | | | | |
| At | 508 | 1.2 | 0.061 | 0 | 1 | 2 |
| start - | | | | | | |
| After 2 | | | | | | |
| months | | | | | | |
| At | 508 | −1.3 | 0.066 | 0 | 1 | 2 |
| start - | | | | | | |
| After 3 | | | | | | |
| months | | | | | | |

TABLE A.2: HEART PALPITATIONS: (SEE ALSO FIG. 2)

| | | | | | | |
|---|---|---|---|---|---|---|
| Heart | | | | | | |
| palpitations | | | | | | |
| At start | 508 | 1.6 | 0.064 | 0 | 1 | 3 |
| After 1 month | 508 | 1.0 | 0.052 | 0 | 1 | 2 |
| After 2 months | 508 | 0.8 | 0.045 | 0 | 0 | 1 |
| After 3 months | 508 | 0.7 | 0.043 | 0 | 0 | 1 |
| At start - | 508 | 0.6 | 0.040 | 0 | 0 | 1 |
| After 1 month * | | | | | | |
| At start - | 508 | 0.8 | 0.047 | 0 | 1 | 1 |
| After 2 months | | | | | | |
| At start - | 508 | 0.9 | 0.051 | 0 | 1 | 2 |
| After 3 months | | | | | | |

TABLE A.3: IRRITABILLITY: (SEE ALSO FIG. 3)

| | | | | | | |
|---|---|---|---|---|---|---|
| Irritability | | | | | | |
| At start | 508 | 3.2 | 0.060 | 2 | 3 | 4 |
| After 1 month | 508 | 2.4 | 0.055 | 1 | 2 | 3 |
| After 2 months | 508 | 2.0 | 0.053 | 1 | 2 | 3 |
| After 3 months | 508 | 1.8 | 0.054 | 1 | 2 | 3 |
| At start - | 508 | 0.8 | 0.042 | 0 | 1 | 1 |
| After 1 month | | | | | | |
| At start - | 508 | 1.1 | 0.049 | 0 | 1 | 2 |
| After 2 months | | | | | | |
| At start - | 508 | 1.3 | 0.053 | 0 | 1 | 2 |
| After 3 months | | | | | | |

TABLE A.4: MOOD SWINGS: (SEE ALSO FIG. 4)

| | | | | | | |
|---|---|---|---|---|---|---|
| Mood Swings | | | | | | |
| At start | 508 | 3.0 | 0.065 | 2 | 3 | 4 |
| After 1 month | 508 | 2.2 | 0.061 | 1 | 2 | 3 |
| After 2 months | 508 | 1.9 | 0.058 | 1 | 2 | 3 |
| After 3 months | 508 | 1.6 | 0.056 | 1 | 1.5 | 2 |
| At start - | 508 | 0.8 | 0.044 | 0 | 1 | 1 |
| After 1 month | | | | | | |
| At start - | 508 | 1.1 | 0.051 | 0 | 1 | 2 |
| After 2 months | | | | | | |
| At start - | 508 | 1.4 | 0.053 | 0.5 | 1 | 2 |
| After 3 months | | | | | | |

TABLE A.5: LOSS OF LIBIDO: (SEE ALSO FIG. 5)

| | | | | | | |
|---|---|---|---|---|---|---|
| Loss of Libido | | | | | | |
| At start | 508 | 3.0 | 0.075 | 2 | 3 | 4 |
| After 1 month | 508 | 2.5 | 0.073 | 1 | 3 | 4 |
| After 2 months | 508 | 2.3 | 0.072 | 1 | 2 | 4 |
| After 3 months | 508 | 2.1 | 0.071 | 1 | 2 | 3 |
| At start - | 508 | 0.5 | 0.040 | 0 | 0 | 1 |
| After 1 month | | | | | | |
| At start - | 508 | 0.7 | 0.047 | 0 | 0 | 1 |
| After 2 months | | | | | | |
| At start - | 508 | 0.9 | 0.052 | 0 | 1 | 2 |
| After 3 months | | | | | | |

TABLE A.6: ANXIETY: (SEE ALSO FIG. 6)

| | | | | | | |
|---|---|---|---|---|---|---|
| Anxiety | | | | | | |
| At start | 508 | 2.9 | 0.068 | 2 | 3 | 4 |
| After 1 month | 508 | 2.2 | 0.064 | 1 | 2 | 3 |
| After 2 months | 508 | 1.9 | 0.060 | 1 | 2 | 3 |
| After 3 months | 508 | 1.7 | 0.060 | 1 | 1 | 3 |
| At start - | 508 | 0.7 | 0.042 | 0 | 1 | 1 |
| After 1 month | | | | | | |
| At start - | 508 | 1.0 | 0.046 | 0 | 1 | 2 |
| After 2 months | | | | | | |
| At start - | 508 | 1.2 | 0.052 | 0 | 1 | 2 |
| After 3 months | | | | | | |

TABLE A.7: MARKED FATIGUE: (SEE ALSO, FIG. 7)

| | | | | | | |
|---|---|---|---|---|---|---|
| Marked fatigue | | | | | | |
| At start | 508 | 3.5 | 0.057 | 3 | 4 | 5 |
| After 1 month | 508 | 2.8 | 0.059 | 2 | 3 | 4 |
| After 2 months | 508 | 2.5 | 0.061 | 1 | 2.5 | 3 |
| After 3 months | 508 | 2.2 | 0.061 | 1 | 2 | 3 |
| At start - | 508 | 0.7 | 0.042 | 0 | 1 | 1 |
| After 1 month | | | | | | |
| At start - | 508 | 1.0 | 0.051 | 0 | 1 | 2 |
| After 2 months | | | | | | |
| At start - | 508 | 1.3 | 0.056 | 0 | 1 | 2 |
| After 3 months | | | | | | |

TABLE A.8: FEELINGS OF DOOM/DREAD (SEE ALSO, FIG. 8)

| | | | | | | |
|---|---|---|---|---|---|---|
| Feelings of | | | | | | |
| doom/dread | | | | | | |
| At start | 508 | 2.4 | 0.076 | 1 | 3 | 4 |
| After 1 month | 508 | 1.8 | 0.067 | 0 | 2 | 3 |
| After 2 months | 508 | 1.4 | 0.061 | 0 | 1 | 2 |
| After 3 months | 508 | 1.3 | 0.059 | 0 | 1 | 2 |
| At start - | 508 | 0.7 | 0.042 | 0 | 0 | 1 |
| After 1 month | | | | | | |
| At start - | 508 | 1.0 | 0.053 | 0 | 1 | 2 |
| After 2 months | | | | | | |
| At start - | 508 | 1.1 | 0.057 | 0 | 1 | 2 |
| After 3 months | | | | | | |

TABLE A.9: VAGINAL DRYNESS: (SEE ALSO, FIG. 9)

| | | | | | | |
|---|---|---|---|---|---|---|
| Vaginal Dryness | | | | | | |
| At start | 508 | 2.0 | 0.073 | 0 | 2 | 3 |
| After 1 month | 508 | 1.7 | 0.068 | 0 | 1 | 3 |
| After 2 months | 508 | 1.5 | 0.063 | 0 | 1 | 2 |
| After 3 months | 508 | 1.3 | 0.060 | 0 | 1 | 2 |
| At start - | 508 | 0.3 | 0.030 | 0 | 0 | 1 |
| After 1 month | | | | | | |
| At start - | 508 | 0.6 | 0.040 | 0 | 0 | 1 |
| After 2 months | | | | | | |
| At start - | 508 | 0.7 | 0.046 | 0 | 0 | 1 |
| After 3 months | | | | | | |

TABLE A.10: INABILITY TO CONCENTRATE: (SEE ALSO, FIG. 10)

| | | | | | | |
|---|---|---|---|---|---|---|
| Inability to | | | | | | |
| Concentrate | | | | | | |
| At start | 508 | 2.8 | 0.608 | 2 | 3 | 4 |
| After 1 month | 508 | 2.3 | 0.059 | 1 | 2 | 3 |
| After 2 months | 508 | 2.0 | 0.056 | 1 | 2 | 3 |
| After 3 months | 508 | 1.8 | 0.055 | 1 | 2 | 3 |
| At start - | 508 | 0.5 | 0.036 | 0 | 0 | 1 |
| After 1 month | | | | | | |
| At start - | 508 | 0.8 | 0.044 | 0 | 1 | 1 |
| After 2 months | | | | | | |
| At start - | 508 | 1.0 | 0.050 | 0 | 1 | 2 |
| After 3 months | | | | | | |

TABLE A.11: TROUBLE SLEEPING: (SEE ALSO, FIG. 11)

| | | | | | | |
|---|---|---|---|---|---|---|
| Trouble | | | | | | |
| Sleeping | | | | | | |
| At start | 508 | 3.7 | 0.061 | 3 | 4 | 5 |
| After 1 month | 508 | 2.8 | 0.066 | 2 | 3 | 4 |
| After 2 months | 508 | 2.4 | 0.067 | 1 | 2 | 4 |
| After 3 months | 508 | 2.2 | 0.067 | 1 | 2 | 3 |
| At start - | 508 | 0.9 | 0.047 | 0 | 1 | 1 |
| After 1 month | | | | | | |
| At start - | 508 | 1.2 | 0.056 | 0 | 1 | 2 |
| After 2 months | | | | | | |
| At start - | 508 | 1.5 | 0.063 | 1 | 1 | 2 |
| After 3 months | | | | | | |

TABLE A.12: URINARY INCONTINENCE: (SEE ALSO FIG. 12)

| | | | | | | |
|---|---|---|---|---|---|---|
| Urinary | | | | | | |
| Incontinence | | | | | | |
| At start | 508 | 2.0 | 0.077 | 0 | 2 | 3 |
| After 1 month | 508 | 1.7 | 0.071 | 0 | 1 | 3 |
| After 2 months | 508 | 1.5 | 0.068 | 0 | 1 | 3 |
| After 3 months | 508 | 1.3 | 0.064 | 0 | 1 | 2 |
| At start - | 508 | 0.4 | 0.036 | 0 | 0 | 1 |
| After 1 month | | | | | | |
| At start - | 508 | 0.6 | 0.046 | 0 | 0 | 1 |
| After 2 months | | | | | | |
| At start - | 508 | 0.7 | 0.050 | 0 | 0 | 1 |
| After 3 months | | | | | | |

TABLE A.13: ITCHY, CRAWLY SKIN: (SEE ALSO FIG. 13)

| | | | | | | |
|---|---|---|---|---|---|---|
| Itchy, Crawly | | | | | | |
| Skin | | | | | | |
| At start | 508 | 1.7 | 0.073 | 0 | 1.5 | 3 |
| After 1 month | 508 | 1.2 | 0.063 | 0 | 1 | 2 |
| After 2 months | 508 | 1.0 | 0.058 | 0 | 0 | 2 |
| After 3 months | 508 | 0.8 | 0.052 | 0 | 0 | 1 |
| At start - | 508 | 0.5 | 0.041 | 0 | 0 | 1 |
| After 1 month | | | | | | |
| At start - | 508 | 0.7 | 0.053 | 0 | 0 | 1 |
| After 2 months | | | | | | |
| At start - | 508 | 0.9 | 0.059 | 0 | 0 | 2 |
| After 3 months | | | | | | |

TABLE A.14: SUDDEN WEIGHT GAIN: (SEE ALSO FIG. 14)

| | | | | | | |
|---|---|---|---|---|---|---|
| Sudden Weight | | | | | | |
| Gain | | | | | | |
| At start | 508 | 2.4 | 0.077 | 1 | 3 | 4 |
| After 1 month | 508 | 2.1 | 0.076 | 0 | 2 | 3 |
| After 2 months | 508 | 1.8 | 0.074 | 0 | 2 | 3 |
| After 3 months | 508 | 1.6 | 0.073 | 0 | 1 | 3 |
| At start - | 508 | 0.3 | 0.043 | 0 | 0 | 1 |
| After 1 month | | | | | | |
| At start - | 508 | 0.6 | 0.052 | 0 | 0 | 1 |
| After 2 months | | | | | | |
| At start - | 508 | 0.8 | 0.058 | 0 | 0.5 | 1 |
| After 3 months | | | | | | |

TABLE A.15: HAIR LOSS: (SEE ALSO FIG. 15)

| | | | | | | |
|---|---|---|---|---|---|---|
| Hair Loss | | | | | | |
| At start | 508 | 1.0 | 0.064 | 0 | 0 | 2 |
| After 1 | 508 | 0.9 | 0.061 | 0 | 0 | 1.5 |
| month | | | | | | |
| After 2 | 508 | 0.8 | 0.055 | 0 | 0 | 1 |
| months | | | | | | |
| After 3 | 508 | 0.7 | 0.053 | 0 | 0 | 1 |
| months | | | | | | |
| At start - | 508 | 0.1 | 0.028 | 0 | 0 | 0 |
| After 1 | | | | | | |
| month | | | | | | |
| At start - | 508 | 0.2 | 0.034 | 0 | 0 | 0 |
| After 2 | | | | | | |
| months | | | | | | |
| At start - | 508 | 0.3 | 0.040 | 0 | 0 | 1 |
| After 3 | | | | | | |
| months | | | | | | |

TABLE A.16: STOMACH PROBLEMS: (SEE ALSO FIG. 16)

| | | | | | | |
|---|---|---|---|---|---|---|
| Stomach | | | | | | |
| Problems | | | | | | |
| At start | 508 | 2.6 | 0.073 | 1 | 3 | 4 |
| After 1 month | 508 | 2.1 | 0.067 | 1 | 2 | 3 |
| After 2 months | 508 | 0.9 | 0.054 | 0 | 0 | 1 |
| After 3 months | 508 | 0.9 | 0.051 | 0 | 0 | 1 |
| At start - | 508 | 0.5 | 0.039 | 0 | 0 | 1 |
| After 1 month | | | | | | |
| At start - | 508 | 1.8 | 0.077 | 0 | 2 | 3 |
| After 2 months | | | | | | |
| At start - | 508 | 1.8 | 0.069 | 0 | 2 | 3 |
| After 3 months | | | | | | |

TABLE A.17: SORE MUSCLES: (SEE ALSO FIG. 17)

| | | | | | | |
|---|---|---|---|---|---|---|
| Painful/Sore | | | | | | |
| Muscles, | | | | | | |
| Tendons, Joints | | | | | | |
| At start | 508 | 2.9 | 0.073 | 2 | 3 | 4 |
| After 1 month | 508 | 2.5 | 0.069 | 1 | 3 | 4 |
| After 2 months | 508 | 2.1 | 0.069 | 1 | 2 | 3 |
| After 3 months | 508 | 1.9 | 0.068 | 0.5 | 2 | 3 |
| At start - | 508 | 0.4 | 0.040 | 0 | 0 | 1 |
| After 1 month | | | | | | |
| At start - | 508 | 0.8 | 0.054 | 0 | 1 | 1 |
| After 2 months | | | | | | |
| At start - | 508 | 1.0 | 0.059 | 0 | 1 | 2 |
| After 3 months | | | | | | |

TABLE A.18: BREAST SORENESS/TENDERNESS: (SEE ALSO FIG. 18)

| | | | | | | |
|---|---|---|---|---|---|---|
| Breast | | | | | | |
| Soreness/ | | | | | | |
| Tenderness | | | | | | |
| At start | 508 | 2.0 | 0.071 | 1 | 2 | 3 |
| After 1 month | 508 | 1.4 | 0.060 | 0 | 1 | 2 |
| After 2 months | 508 | 1.2 | 0.055 | 0 | 1 | 2 |
| After 3 months | 508 | 1.0 | 0.053 | 0 | 1 | 2 |
| At start - | 508 | 0.6 | 0.046 | 0 | 0 | 1 |
| After 1 month | | | | | | |
| At start - | 508 | 0.8 | 0.055 | 0 | 1 | 1 |
| After 2 months | | | | | | |
| At start - | 508 | 1.0 | 0.062 | 0 | 1 | 2 |
| After 3 months | | | | | | |

TABLE A.19: IRREGULAR VAGINAL BLEEDING: (SEE ALSO FIG. 19)

| | | | | | | |
|---|---|---|---|---|---|---|
| Irregular | | | | | | |
| vaginal | | | | | | |
| Bleeding | | | | | | |
| At start | 508 | 1.8 | 0.079 | 0 | 1 | 3 |
| After 1 month | 508 | 1.2 | 0.070 | 0 | 0 | 2 |
| After 2 months | 508 | 0.9 | 0.058 | 0 | 0 | 2 |
| After 3 months | 508 | 0.7 | 0.055 | 0 | 0 | 1 |
| At start - | 508 | 0.5 | 0.052 | 0 | 0 | 1 |
| After 1 month | | | | | | |
| At start - | 508 | 0.9 | 0.062 | 0 | 0 | 2 |
| After 2 months | | | | | | |
| At start - | 508 | 1.0 | 0.067 | 0 | 0 | 2 |
| After 3 months | | | | | | |

TABLE A.20: LAPSES OF MEMORY: (SEE ALSO FIG. 20)

| | | | | | | |
|---|---|---|---|---|---|---|
| Disturbing | | | | | | |
| Memory Lapses | | | | | | |
| At start | 508 | 2.7 | 0.064 | 2 | 3 | 4 |
| After 1 month | 508 | 2.3 | 0.062 | 1 | 2 | 3 |
| After 2 months | 508 | 2.0 | 0.060 | 1 | 2 | 3 |
| After 3 months | 508 | 1.8 | 0.059 | 1 | 2 | 3 |
| At start - | 508 | 0.4 | 0.037 | 0 | 0 | 1 |
| After 1 month | | | | | | |
| At start - | 508 | 0.7 | 0.047 | 0 | 1 | 1 |
| After 2 months | | | | | | |
| At start - | 508 | 1.0 | 0.051 | 0 | 1 | 2 |
| After 3 months | | | | | | |

TABLE A.21: MUSCLE TENSION: (SEE ALSO FIG. 21)

| | | | | | | |
|---|---|---|---|---|---|---|
| Increased | | | | | | |
| Muscle Tension | | | | | | |
| At start | 508 | 2.3 | 0.069 | 1 | 3 | 3 |
| After 1 month | 508 | 1.9 | 0.064 | 0 | 2 | 3 |
| After 2 months | 508 | 1.6 | 0.060 | 0 | 1 | 3 |
| After 3 months | 508 | 1.4 | 0.058 | 0 | 1 | 2 |
| At start - | 508 | 0.5 | 0.036 | 0 | 0 | 1 |
| After 1 month | | | | | | |
| At start - | 508 | 0.7 | 0.048 | 0 | 0 | 1 |
| After 2 months | | | | | | |
| At start - | 508 | 0.9 | 0.055 | 0 | 1 | 2 |
| After 3 months | | | | | | |

TABLE A.22: PAINFUL INTERCOURSE: (SEE ALSO FIG. 22)

| | | | | | | |
|---|---|---|---|---|---|---|
| Painful | | | | | | |
| Intercourse | | | | | | |
| At start | 508 | 1.3 | 0.069 | 0 | 1 | 2 |
| After 1 month | 508 | 1.0 | 0.063 | 0 | 0 | 2 |
| After 2 months | 508 | 0.9 | 0.058 | 0 | 0 | 1 |
| After 3 months | 508 | 0.8 | 0.054 | 0 | 0 | 1 |
| At start - | 508 | 0.3 | 0.029 | 0 | 0 | 0 |
| After 1 month | | | | | | |
| At start - | 508 | 0.4 | 0.039 | 0 | 0 | 1 |
| After 2 months | | | | | | |
| At start - | 508 | 0.6 | 0.043 | 0 | 0 | 1 |
| After 3 months | | | | | | |

TABLE A.23: BLADDER INFECTIONS: (SEE ALSO FIG. 23)

| | | | | | | |
|---|---|---|---|---|---|---|
| Bladder | | | | | | |
| Infections | | | | | | |
| At start | 508 | 0.7 | 0.055 | 0 | 0 | 1 |
| After 1 month | 508 | 0.4 | 0.044 | 0 | 0 | 0 |
| After 2 months | 508 | 0.3 | 0.035 | 0 | 0 | 0 |
| After 3 months | 508 | 0.2 | 0.027 | 0 | 0 | 0 |
| At start - | 508 | 0.3 | 0.036 | 0 | 0 | 0 |
| After 1 month | | | | | | |
| At start - | 508 | 0.4 | 0.043 | 0 | 0 | 1 |
| After 2 months | | | | | | |
| At start - | 508 | 0.5 | 0.048 | 0 | 0 | 1 |
| After 3 months | | | | | | |

Appendix 2. HORMONE LEVELS

| | | | | |
|---|---|---|---|---|
| | Standard error | | | |
| Mean | (mean) | p25 | p50 | p75 |

TABLE A.24: IGF-1 (SEE ALSO FIG. 24)

| | | | | | | |
|---|---|---|---|---|---|---|
| igf1 | At start | 17.49 | 1.294729 | 13.7 | 16.7 | 20.2 |
| igf2 | After 2 | 16.36 | 1.552002 | 11 | 15.85 | 20 |
| | months | | | | | |
| d_igf | 2-0 | 1.13 | 0.9262889 | −0.5 | 1.45 | 2.7 |

TABLE A.25: TSH (SEE ALSO FIG. 25)

| | | | | | | |
|---|---|---|---|---|---|---|
| tsh | At start | 2.451 | 0.6367495 | 1.37 | 1.785 | 2.44 |
| tsh2 | After 2 | 2.232 | 0.6354435 | 1.26 | 1.49 | 1.74 |
| | months | | | | | |
| d_tsh | 2-0 | 0.219 | 0.1235084 | −0.04 | 0.165 | 0.35 |

TABLE A.26: FREE THYROXINE (SEE ALSO FIG. 26)

| | | | | | | |
|---|---|---|---|---|---|---|
| thyr1 | At start | 16.24 | 0.7248294 | 14.8 | 17 | 18.1 |
| thyr2 | After 2 | 16 | 0.6764286 | 15 | 16.65 | 17.4 |
| | months | | | | | |
| d_thyr | 2-0 | 0.2399997 | 0.284097 | −0.2000008 | −0.0500007 | 1.1 |

TABLE A.27: FREE T3 (SEE ALSO FIG. 27)

| | | | | | | |
|---|---|---|---|---|---|---|
| t31 | At start | 4.29 | 0.1754043 | 4.2 | 4.5 | 4.6 |
| t32 | After 2 | 4.64 | 0.1439135 | 4.4 | 4.5 | 5 |
| | months | | | | | |
| d_t3 | 2-0 | −0.3500001 | 0.1424001 | −0.5999999 | −0.3500001 | 0.0999999 |

TABLE A.28: FSH (SEE ALSO FIG. 28)

| | | | | | | |
|---|---|---|---|---|---|---|
| fsh1 | At start | 96.31 | 9.96656 | 76.8 | 88.95 | 111.9 |
| fsh2 | After 2 | 108.13 | 7.85246 | 86.4 | 101.85 | 120.9 |
| | months | | | | | |
| d_fsh | 2-0 | −11.82 | 6.513419 | −12.6 | −10.4 | 2.100006 |

TABLE A.29: LH (SEE ALSO FIG. 29) (STATISTICALLY
SIGNIFICANT CHANGE)

| | | | | | | |
|---|---|---|---|---|---|---|
| 1h1 | At start | 46.56 | 4.179187 | 40 | 43.45 | 54.7 |
| 1h2 | After 2 | 51.26 | 4.059042 | 42 | 48.45 | 58.6 |
| | months | | | | | |
| d_1h | 2-0 | −4.699999 | 2.162355 | −4.599998 | −2.950001 | −2 |

TABLE A.30: PROGESTERONE (SEE ALSO FIG. 30)

| | | | | | | |
|---|---|---|---|---|---|---|
| prog1 | At start | 1 | 0.1849925 | 0.5 | 0.9 | 1.2 |
| prog2 | After 2 | 1.02 | 0.2009975 | 0.5 | 0.7 | 1.5 |
| | months | | | | | |
| d_prog1 | 2-0 | −0.02 | 0.0553775 | 0 | 0 | 0 |

TABLE A.31: TESTOSTERONE (SEE ALSO FIG. 31)

| | | | | | | |
|---|---|---|---|---|---|---|
| test1 | At start | 0.77 | 0.1591994 | 0.4 | 0.7 | 0.9 |
| test2 | After 2 | 0.98 | 0.1884439 | 0.5 | 0.8 | 1.3 |
| | months | | | | | |
| d_test | 2-0 | −0.21 | 0.1015983 | −.5999999 | −0.1 | −0.1 |

TABLE A.32: SHBG (SEE ALSO FIG. 32)

| | | | | | | |
|---|---|---|---|---|---|---|
| shbg1 | At start | 60.1 | 7.469122 | 41 | 62 | 79 |
| shbg2 | After 2 | 61.2 | 8.41797 | 40 | 65.5 | 74 |
| | months | | | | | |
| d_shbg | 2-0 | −1.1 | 3.634556 | −1 | 0.5 | 5 |

TABLE A.33: ESTRADIOL (SEE ALSO FIG. 33) (STATISTICALLY
SIGNIFICANT CHANGE)

| | | | | | | |
|---|---|---|---|---|---|---|
| estr1 | At start | 93.1 | 29.57982 | 60 | 65 | 73 |
| estr2 | After 2 | 45.4 | 0.9451631 | 44 | 44 | 44 |
| | months | | | | | |
| d_estr | 2-0 | 47.7 | 29.75046 | 10 | 18 | 29 |

TABLE A.34: DHEA (SEE ALSO FIG. 34)

| | | | | | | |
|---|---|---|---|---|---|---|
| dhea1 | At start | 4.66 | 0.9045809 | 3 | 3.6 | 5.5 |
| dhea2 | After 2 | 4.56 | 0.8523432 | 2.9 | 3.55 | 6.6 |
| | months | | | | | |
| d_dhea | 2-0 | 0.1 | 0.246306 | −0.1999999 | 0.3 | 0.5000001 |

TABLE A.35: CORTISOL (SEE ALSO FIG. 35)

| | | | | | | |
|---|---|---|---|---|---|---|
| cort1 | At start | 209.7 | 25.21157 | 146 | 192.5 | 249 |
| cort2 | After 2 | 262.9 | 44.40557 | 195 | 252 | 315 |
| | months | | | | | |
| d_cort | 2-0 | −53.2 | 28.26415 | −123 | −65 | 17 |

TABLE A.36: FREE TESTOSTERONE (SEE ALSO FIG. 36)

| | | | | | | |
|---|---|---|---|---|---|---|
| ftesto1 | At start | 0.579 | 0.0980867 | 0.44 | 0.47 | 0.8 |
| ftesto2 | After 2 | 0.697 | 0.1579877 | 0.4 | 0.555 | 0.84 |
| | months | | | | | |
| d_ftesto | 2-0 | −0.118 | 0.099463 | −0.22 | −0.035 | 0.06 |

TABLE A.37: ESTRONE (SEE ALSO FIG. 37)

| | | | | | | |
|---|---|---|---|---|---|---|
| estro1 | At start | 0.162 | 0.0198214 | 0.11 | 0.15 | 0.2 |
| estro2 | After 2 | 0.135 | 0.0174642 | 0.09 | 0.135 | 0.16 |
| | months | | | | | |
| d_estro | 2-0 | 0.027 | 0.0174515 | 0 | 0.02 | 0.03 |

TABLE A.38: ESTRIOL (SEE ALSO FIG. 38) STATISTICALLY
SIGNIFICANT CHANGE

| | | | | | | |
|---|---|---|---|---|---|---|
| estri1 | At start | 0.143 | 0.0359026 | 0.04 | 0.11 | 0.24 |
| estri2 | After 2 | 0.319 | 0.0236385 | 0.26 | 0.3 | 0.4 |
| | months | | | | | |
| d_estri | 2-0 | −0.176 | 0.0485157 | −0.29 | −0.205 | −0.07 |

TABLE A.39: IGFBP-3 (SEE ALSO FIG. 39) STATISTICALLY
SIGNIFICANT CHANGE

| | | | | | | |
|---|---|---|---|---|---|---|
| igfbp1 | At start | 3.782 | 0.3444796 | 3.01 | 3.415 | 4.54 |
| igfbp2 | After 2 | 4.237 | 0.2565153 | 3.51 | 4.175 | 4.88 |
| | months | | | | | |
| d_igfbp | 2-0 | −0.4550001 | 0.1650404 | −1.01 | −0.3100002 | −0.1500001 |

1. (canceled)

2. A method of treating and/or preventing cancer comprising applying a magnetic product to a human or animal body wherein the magnetic product comprises a magnet having positive and negative poles, the magnet having an additional metallic element which distorts the magnetic field distribution around the magnet so as to accentuate the magnetic field in the vicinity of the negative pole of the magnet.

3. The method according to claim 2 wherein the cancer is a breast cancer, prostate cancer, lung cancer, melanoma, head or neck cancer.

4. The method according to claim 1 wherein the cancer is a female specific cancer.

5. The method according to any one of claims 2, 3 or 4 wherein the method increases the level of IGFBP-3 in a subject wearing the magnetic product compared to a base level established in the subject prior to commencement of wearing the magnetic product and/or compared to a base level established in a control subject, or group of subjects.

6. (canceled)

7. A method of increasing the level of IGFBP-3 in a subject comprising applying a magnetic product to a subject wherein the magnetic product comprises a magnet having positive and negative poles, the magnet having an additional metallic element which distorts the magnetic field distribution around the magnet so as to accentuate the magnetic field in the vicinity of the negative pole of the magnet.

8. The method according to claim 2 or 7 wherein the method use also increases the level of luteinising hormone and/or decreases the level of estradiol and/or increases the level of estriol in the subject.

9. The method according to claim 5 wherein the control subject is a male or a non-menopausal female.

10. (canceled)

11. (canceled)

12. The or method according to claim 2 or 7 wherein the magnetic product is applied to the pelvic region of a female human.

13. The or method according to any one of claim 2 or 7 wherein the magnetic product is applied to the body for at least 2, 4, 6, 8, 10, 12, 14, 16, 18, 20, 22 or 24 hours/day.

14. The or method according to any one of claim 2 or 7 wherein the magnetic product is applied to the body for from 7 to 28 days, 7 to 14 days or 7 to 21 days.

15. The method according to any one of claim 2 or 7 wherein the magnetic product comprises a plurality of magnets.

16. The method according to claim 15 wherein the magnetic product comprises four magnets.

17. The or method according to any of claims 2 or 7 wherein one or more of the magnets has a strength of from about 1,800 G to about 2,800 G.

18. The method according to any of claims 2 or 7 wherein one or more of the magnets comprises neodymium

19. (canceled)
